(19) United States
(12) Patent Application Publication
(10) Pub. No.: US 2026/0108133 A1
(43) Pub. Date: Apr. 23, 2026

(54) ENDOSCOPIC DEVICE AND METHOD FOR DIAGNOSING GASTRIC LESION BASED ON GASTROENDOSCOPIC IMAGES ACQUIRED IN REAL TIME

(71) Applicant: INDUSTRY ACADEMIC COOPERATION FOUNDATION, HALLYM UNIVERSITY, Chuncheon-si (KR)

(72) Inventors: Bum-Joo CHO, Seoul (KR); Chang Seok BANG, Chuncheon-si (KR); Se Woo PARK, Hwaseong-si (KR); Jae-Jun LEE, Chuncheon-si (KR); Jae-Ho CHOI, Chuncheon-si (KR)

(21) Appl. No.: 17/278,983

(22) PCT Filed: Sep. 25, 2019
(86) PCT No.: PCT/KR2019/012449

§ 371 Date: Jan. 2, 2026

(30) Foreign Application Priority Data
Oct. 2, 2018 (KR) 10-2018-0117824

(51) Int. Cl. A61B 1/00; G06T 7/00; G16H 50/20

(52) U.S. Cl. CPC A61B 1/000094; A61B 1/00045; G06T 7/0012; G16H 50/20; G06T 2207/20081; G06T 2207/30092

(57) ABSTRACT

An endoscopic device for diagnosing a gastric lesion from gastroendoscopic images acquired in real time is provided. The endoscopic device includes: a body part containing a plurality of units, that is inserted into a patient's body; an operation part provided on the rear end of the body part, for manipulating the body part based on user input information; a lesion diagnostic part that builds an artificial neural network system by training the artificial neural network system by using a plurality of gastric lesion images as input and gastric lesion diagnostic results as output, generates a new dataset by linking new gastroendoscopic images acquired in real time with patient information, and performs a gastric lesion diagnosis through the built artificial neural network system; and a display that shows the diagnostic results of the lesion diagnostic part and the new gastroendoscopic images acquired in real time.

### CROSS-REFERENCE TO RELATED APPLICATIONS

This application claims the benefit under 35 U.S.C. section 371, of PCT International Application No.: PCT/KR2019/012449, filed on Sep. 25, 2019, which claims foreign priority to Korean Patent Application No.: 10-2018-0117824, filed on Oct. 2, 2018, in the Korean Intellectual Property Office, both of which are hereby incorporated by reference in their entireties.

### BACKGROUND OF THE DISCLOSURE

### Field of the Disclosure

The present disclosure relates to an endoscopic device and method for diagnosing a gastric lesion based on gastroendoscopic images acquired in real time.

### Related Art

Cells, the smallest units that make up the human body, divide by intracellular regulatory functions when normal, and maintain cell balance while growing, dying, and disappearing. When a cell is damaged for some reason, it is treated and regenerated to serve as a normal cell, but if it does not recover, it will die by itself. However, cancer is defined as a condition in which abnormal cells that do not control proliferation and inhibition for many reasons are not only excessively proliferating but also invade surrounding tissues and organs, resulting in mass formation and normal tissue destruction. Cancer is a cell proliferation that cannot be inhibited, and it destroys normal cell and organ structure and function, so its diagnosis and treatment are very important.

Cancer is a group of diseases in which cells proliferate infinitely and interfere with normal cellular functions, the most common examples of which are lung cancer, gastric cancer (GC), breast cancer (BRC), and colorectal cancer (CRC) though they can develop in virtually any tissue. Of these cancers, globally, gastric cancer is more prevalent in South Korea and Japan, whereas the incidence rates of gastric cancer are rather low in Western countries such as the United States and Europe. In South Korea, gastric cancer is the cancer of highest incidence and also the second leading cause of cancer death after lung cancer, which means that gastric cancer is one of the most common forms of cancer that affects national health. As for types of gastric cancer, 95% of gastric cancers are adenocarcinomas, which originate in glandular cells of the mucosa that lines the stomach. Other cancers include lymphoma, which originates in the lymphatic system, and gastrointestinal stromal tumor, which originates in stromal tissues. Most early gastric cancers (ECG) cause no clinical symptoms or signs, which make it difficult to detect and treat them at the right time without a screening strategy. Moreover, patients with premalignant lesions such as dysplasia are at high risk of gastric cancer.

The most common methods used to diagnose gastric cancer are to use a tissue sample taken by a biopsy, perform gastroendoscopy, or use computed tomography (CT) images or nuclear magnetic resonance (NMR) images. Among these, the biopsy is disadvantageous in that it causes great pain to the patient and is not only expensive but also takes a long time to diagnose. Also, biopsies are invasive examinations that may damage the patient's tissue. In addition, if a patient actually has cancer, cancer metastasis may be induced during the biopsy process, and therefore repeat invasive biopsies may carry a risk of harm to the patient. Diagnoses using computed tomography or nuclear magnetic resonance may cause misdiagnosis depending on the skill level of a clinician or an interpreting physician, and greatly depends on the accuracy of the device that acquires the images. Furthermore, even the most accurate devices cannot detect tumors as small as or smaller than several mm, which makes it difficult to detect cancer in the early stages of development. Also, in order to obtain a picture, the patient or disease holder is exposed to high-energy electromagnetic waves that can induce gene mutations, which may cause other diseases as well.

For this reason, in the past, neoplasms of the stomach were usually discovered when a doctor performs gastroendoscopy, and identified as cancers primarily based on their shapes and sizes in the stomach as observed on endoscopic images. For a lesion suspicious for cancer, a sample tissue was taken by gastroendoscopy, and the lesion was confirmed as cancer by a pathological biopsy. However, gastroendoscopy requires the patient to swallow an endoscope down their throat, which causes a lot of discomfort as it reaches the stomach through the esophagus, and there are risks of complications such as perforation of the stomach or esophagus. Thus, it is crucial to reduce the number of repeat biopsies in diagnosing neoplasms of the stomach for the sake of the patient.

Therefore, it is very important to assess the risk of cancer in real time upon discovering neoplastic gastric lesions from gastroendoscopic images during a one-time gastroendoscopic procedure, make a decision immediately whether to perform a biopsy or not on a certain lesion, and then proceed directly to doing a biopsy on a lesion suspicious for cancer, rather than a doctor performing gastroendoscopy for the discovery of neoplasms of the stomach, analyzing the results, and then repeating gastroendoscopy prior to a biopsy. That is, the current tendency is to gradually reduce the number of repeat gastroendoscopic procedures. In assessing the risk of neoplastic gastric lesions in real time, if the assessed level of risk is lower than the actual risk, cancerous lesions will be left undetected, leading to the patient not getting adequate cancer treatment, and if the assessed level of risk is higher than the actual risk, unnecessary biopsies may be done on the patient and therefore cause harm to the patient's tissue.

However, there is no standardized method of assessing the risk of gastric lesions from real-time gastroendoscopic images that has been established so far. At the present, risk assessment relies entirely on a subjective decision by a doctor who performs endoscopy. This method, however, will produce different diagnoses depending on the doctor's experience and does not ensure accurate diagnosis in areas where there are no doctors with sufficient experience.

The detection of abnormal lesions is usually based on abnormal morphology or color changes in the mucosa, and it is known that diagnostic accuracy is improved through learning and optical techniques or chromoendoscopy. The application of endoscopic imaging technologies such as narrow-band imaging, confocal imaging or magnifying techniques (so-called image-enhanced endoscopy) is also known to enhance diagnostic accuracy.

However, examination solely with white-light endoscopy remains the most routine form of screening, and standardization of the procedure and improvements in the interpretation process to resolve the interobserver and intraobserver variability are needed in image-enhanced endoscopy.

The related art to the present disclosure is disclosed in Korean Unexamined Patent Publication No. 10-2018-0053957.

### SUMMARY

The present disclosure has been made in an effort to solve the aforementioned problems occurring in the related art and to provide an endoscopic device which can diagnose a gastric lesion in real time during gastroendoscopy by collecting white-light gastroendoscopic images acquired by an endoscopic video imaging device and feeding them in real time into a deep learning algorithm.

The present disclosure has been made in an effort to solve the aforementioned problems occurring in the related art and to provide an endoscopic device which provides a deep learning model for automatically classifying gastric tumors based on gastroendoscopic images.

The present disclosure has been made in an effort to solve the aforementioned problems occurring in the related art and to provide an endoscopic device which can diagnose a barely noticeable gastric tumor by evaluating multiple image data in real time which is acquired when a doctor (user) examines the gastric tumor using an endoscopic device.

The present disclosure has been made in an effort to solve the aforementioned problems occurring in the related art and to provide an endoscopic device which can diagnose and predict gastric cancer or gastric dysplasia by automatically classifying a neoplasm of the stomach based on gastroendoscopic images acquired in real time.

However, the technical problems to be solved in the present disclosure are not limited to the above-described ones, and other technical problems may be present.

As a technical means for solving the above-mentioned technical problems, an exemplary embodiment of the present disclosure provides an endoscopic device for diagnosing a gastric lesion from gastroendoscopic images acquired in real time, the endoscopic device including: a body part containing a plurality of units, that is inserted into a patient's body; an operation part provided on the rear end of the body part, for manipulating the body part based on user input information; a lesion diagnostic part that builds an artificial neural network system by training the artificial neural network system by using a plurality of gastric lesion images as input and gastric lesion diagnostic results as output, generates a new dataset by linking new gastroendoscopic images with patient information, and performs a gastric lesion diagnosis through the built artificial neural network system; and a display that shows the diagnostic results of the lesion diagnostic part and the new gastroendoscopic images acquired in real time.

According to an exemplary embodiment of the present disclosure, the endoscopic device may further include a controller that generates a control signal for controlling the operation of the body part based on the user input information provided from the operation part and the diagnostic results of the lesion diagnostic device.

According to an exemplary embodiment of the present disclosure, the body part may include an imaging part provided on the front end of the body part, that takes new gastric lesion images and provides the taken, new gastroendoscopic images to the lesion diagnostic part, wherein the controller may receive the user's input for controlling the operation of the imaging part from the operation part and generate a control signal for controlling the imaging part.

According to an exemplary embodiment of the present disclosure, the endoscopic device may further include a lesion location acquisition part that generates gastric lesion information by linking the new gastroendoscopic images provided from the imaging part with location information, wherein the controller may generate a control signal for controlling the operation of a biopsy unit for sampling a portion of tissue in the patient's body based on the diagnostic results of the lesion diagnostic part and the gastric lesion information.

According to an exemplary embodiment of the present disclosure, the lesion diagnostic part may include: an image acquisition part for acquiring the new gastric lesion images; a data generation part for generating a new dataset by linking the new gastric lesion images with the patient information; a data preprocessing part for preprocessing the new dataset in a way that is applicable to a deep learning algorithm; an artificial neural network building part for building an artificial neural network system by training the artificial neural network system by using a plurality of gastric lesion images as input and gastric lesion diagnostic results as output; and a gastric lesion diagnostic part that performs a gastric lesion diagnosis through the artificial neural network system after passing the new dataset through a preprocessing process.

According to an exemplary embodiment of the present disclosure, the data generation part may generate a dataset by linking each of the plurality of gastric lesion images with the patient information, and the dataset, when generated, may be classified as a training dataset required for training the artificial neural network system or a validation dataset for validating information on the progress of the training of the artificial neural network system.

According to an exemplary embodiment of the present disclosure, the validation dataset may be a dataset that is not redundant with the training dataset.

According to an exemplary embodiment of the present disclosure, the preprocessing part may preprocess a gastric lesion image in the new dataset in a way that is applicable to the deep learning algorithm by performing at least one of the following phases of the preprocessing process: cropping a peripheral area of the gastric lesion image around the gastric lesion in such a way that the gastric lesion is not included in the image; shifting the gastric lesion image in parallel upward, downward, to the left, or to the right; rotating the gastric lesion image; flipping the gastric lesion image; and adjusting colors in the gastric lesion image.

According to an exemplary embodiment of the present disclosure, the preprocessing part may include an augmentation part for increasing the amount of new gastric lesion image data, wherein the augmentation part may augment the new gastric lesion image data by applying at least one of the following: rotating, flipping, cropping, and adding noise into the gastric lesion image data.

According to an exemplary embodiment of the present disclosure, the artificial neural network building part may build a training model in which a convolutional neural network and a fully-connected neural network are trained by using the preprocessed dataset as input and the gastric lesion diagnostic results as output.

According to an exemplary embodiment of the present disclosure, the preprocessed dataset may be fed as input into the convolutional neural network, and the output of the convolutional neural network and the patient information may be fed as input into the fully-connected neural network.

According to an exemplary embodiment of the present disclosure, the convolutional neural network may produce a plurality of feature patterns from the plurality of gastric lesion images, wherein the plurality of feature patterns may be finally classified by the fully-connected neural network.

According to an exemplary embodiment of the present disclosure, the gastric lesion diagnostic part may classify the gastric lesion diagnosis as at least one of the following categories: advanced gastric cancer, early gastric cancer, high-grade dysplasia, and low-grade dysplasia.

An exemplary embodiment of the present disclosure provides a method for diagnosing a gastric lesion from gastroendoscopic images acquired in real time by an endoscopic device, the endoscopic device including: a body part to be inserted into a patient's body; and an operation part provided on the rear end of the body part, for manipulating the body part based on user input information, the method including: building an artificial neural network system by training the artificial neural network system by using a plurality of gastric lesion images as input and gastric lesion diagnostic results as output, generating a new dataset by linking new gastroendoscopic images acquired in real time with patient information, and performing a gastric lesion diagnosis through the built artificial neural network system; and showing the diagnostic results and the new gastroendoscopic images acquired in real time.

The above-mentioned solutions are merely exemplary and should not be construed as limiting the present disclosure. In addition to the above-described exemplary embodiments, additional embodiments may exist in the drawings and detailed description of the disclosure.

According to the above-described means for solving the problems of the present disclosure, it is possible to diagnose a gastric lesion by collecting white-light gastroendoscopic images acquired with an endoscopic video imaging device and feeding them into a deep learning algorithm.

According to the above-described means for solving the problems of the present disclosure, it is possible to provide a deep learning model for automatically classifying gastric tumors based on gastroendoscopic images.

According to the above-described means for solving the problems of the present disclosure, it is possible to diagnose a barely noticeable gastric tumor by learning multiple image data in real time which is acquired when a doctor (user) examines the gastric tumor using an endoscopic device.

According to the above-described means for solving the problems of the present disclosure, it is possible to significantly reduce cost and labor, compared with the existing gastroendoscopy which requires a doctor's experience, by learning images acquired with an endoscopic video imaging device and classifying gastric lesions.

According to the above-described means for solving the problems of the present disclosure, it is possible to obtain objective and consistent interpretation and reduce potential mistakes and misinterpretation by an interpreting doctor, since a gastric lesion can be diagnosed and predicted with the above gastric lesion diagnostic device based on gastroendoscopic images acquired with an endoscopic video imaging device, and it is also possible to use the above gastric lesion diagnostic device as an aid for clinical decision.

However, advantageous effects to be achieved in the present disclosure are not limited to the above-described ones, and other advantageous effects may be present.

### BRIEF DESCRIPTION OF THE DRAWINGS

FIG. 1 is a schematic diagram of an endoscopic device according to an exemplary embodiment of the present disclosure.

FIG. 2 is a schematic block diagram of an endoscopic device according to an exemplary embodiment of the present disclosure.

FIG. 3 is a schematic block diagram of a lesion diagnostic part of an endoscopic device according to an exemplary embodiment of the present disclosure.

FIG. 4 is an operation flow chart of a method for diagnosing a lesion from gastroendoscopic images acquired in real time by an endoscopic device according to an exemplary embodiment of the present disclosure.

### DESCRIPTION OF EXEMPLARY EMBODIMENTS

Hereinafter, embodiments of the present disclosure will be described in detail with reference to the accompanying drawings so that those skilled in the art can easily carry out the present disclosure. It should be understood, however, that the present disclosure may be embodied in many different forms and should not be construed as limited to the embodiments set forth herein. In the drawings, the same reference numbers are used throughout the specification to refer to the same or like parts.

Throughout this specification, it will be understood that, when a certain portion is referred to as being “connected” to another portion, this means not only that the certain portion is “directly connected” to the another portion, but also that the certain portion is “electrically connected” or “indirectly connected” to the another portion with an intervening element therebetween.

Throughout this specification, it will be understood that, when a certain member is located “on”, “above”, “on the top of”, “under”, “below”, or “on the bottom of” another member, this means not only that the certain member comes into contact with the another member, but also that there is an intervening member between the two members.

Throughout this specification, it will be understood that, when a certain portion “includes” a certain element, this does not preclude the presence of another element but the certain portion may include another element unless the context clearly dictates otherwise.

The present disclosure relates to a gastric lesion diagnostic device and method including a deep learning model for classifying gastric tumors based on gastroendoscopic images acquired from an endoscopic device and evaluating the performance of the device. The present disclosure allows for automatically diagnosing a neoplasm of the stomach by interpreting gastroendoscopic images based on a convolutional neural network.

The present disclosure enables the diagnosis and prediction of gastric cancer or gastric dysplasia by computer-training a convolutional neural network, which is a type of deep learning algorithm, on a dataset of gastroendoscopic picture images, interpreting newly input gastroendoscopic pictures, and therefore automatically classifying a neoplasm of the stomach in the pictures.

The present disclosure enables the diagnosis and prediction of gastric cancer or gastric dysplasia by interpreting new gastroendoscopic images acquired in real time by an artificial neural network system built based on a plurality of gastric lesion images.

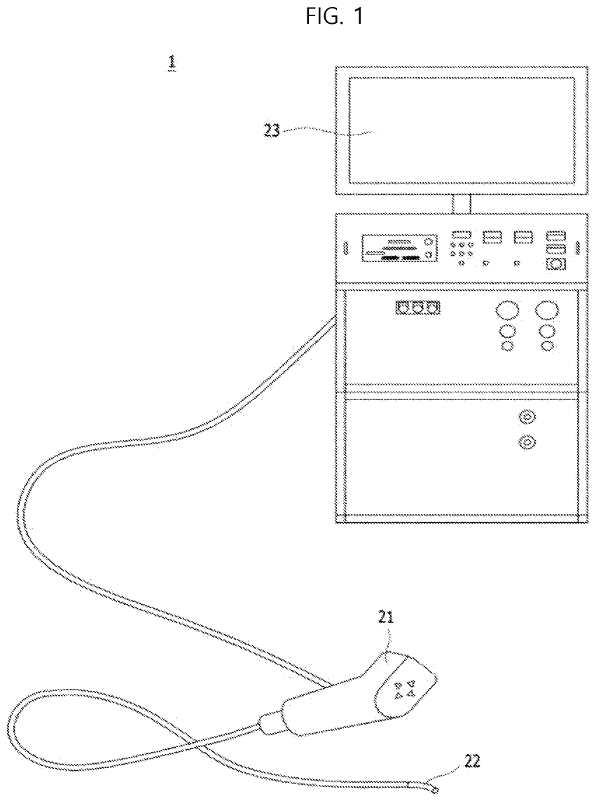

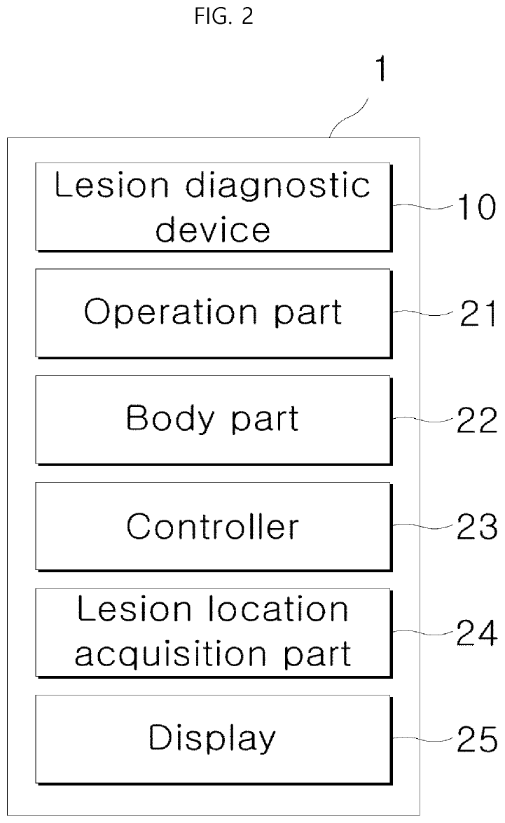

FIG. 1 is a schematic diagram of an endoscopic device according to an exemplary embodiment of the present disclosure. FIG. 2 is a schematic block diagram of an endoscopic device according to an exemplary embodiment of the present disclosure.

Referring to FIGS. 1 and 2, the endoscopic device 1 may include an operation part 21, a body part 22, a controller 23, a lesion location acquisition part 24, and a display 25.

The endoscopic device 1 may send and receive data (images, video, and text) and a variety of communication signals over a network. The endoscopic device 1 may be comprised of variety types of servers, terminals, or devices having data storage and processing functions.

The endoscopic device 1 may be a device used for gastroendoscopic examination. As illustrated in FIG. 1. the endoscopic device 1 may include an operation part 21 to manipulate the body part 22 based on user input information. Also, the endoscopic device 1 may be made in capsule form. The capsule endoscopic device 1 may include a miniature camera, which may be inserted into a patient's body to acquire gastroendoscopic images. The endoscopic device 1 is not limited to the above-mentioned form.

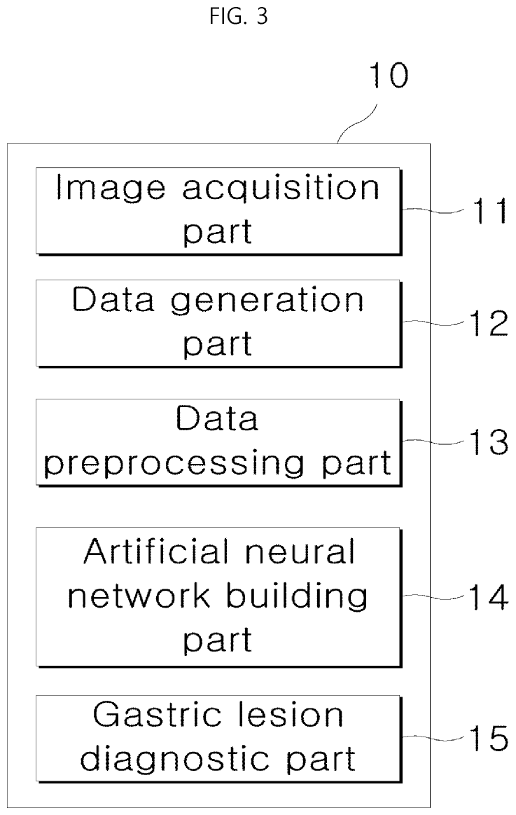

The lesion diagnostic part 10 may build an artificial neural network system by training it by using a plurality of gastric lesion images as input and gastric lesion diagnostic results as output, generate a new dataset by linking the gastric lesion images with patient information, and perform a gastric lesion diagnosis through the built artificial neural network system. In other words, the lesion diagnostic part 10 may perform a gastric lesion diagnosis after learning real-time gastric lesion images through the built artificial neural network system. The lesion diagnostic part 10 will be described in more details with reference to FIG. 3 to be described later.

According to another exemplary embodiment of the present disclosure, the operation part 21 may be provided on the rear end of the body part 22 and manipulated based on information inputted by the user. The operation part 21 is a part that is gripped by an endoscopist, with which the body part 22 to be inserted into the patient's body. Also, the operation part 21 allows for manipulating the operation of a plurality of units required for an endoscopic procedure the body part 22 contains. The operation part 21 may include a rotary controller. The rotary controller may include a part that functions to generate a control signal and provides rotational force (such as in a motor). The operation part 21 may include buttons for manipulating the imaging part (not shown). The buttons are used to control the position of the imaging part (not shown), by which the user may change the position of the body part 22 upward, downward, to the left, to the right, forward, backward, and so forth.

The body part 22 is a part that is inserted into the patient's body, and may contain a plurality of units. The plurality of units may include at least one of an imaging part (not shown) for imaging the inside of the patient's body, an air supply unit for supplying air into the body, a water supply unit for supplying water into the body, a lighting unit for illuminating the inside of the body, a biopsy unit for sampling a portion of tissue in the body or treating the tissue, and a suction unit for sucking air or foreign materials from inside the body. The biopsy unit may include a variety of medical instruments, such as scalpels, needles, and so on, for sampling a portion of tissue from a living organism, and the scalpels and needles in the biopsy unit may be inserted into the body through a biopsy channel by the endoscopist to sample cells in the body.

The imaging part (not shown) may hold a camera of a size equivalent to the diameter of the body part 22. The imaging part (not shown) may be provided on the front end of the body part 22 and take gastric lesion images and provide the taken gastric lesion images to the lesion diagnostic part 10 and the display 25 over a network. The imaging part (not shown) may acquire new gastric lesion images in real time.

The controller 23 may generate a control signal for controlling the operation of the body part 22 based on the user input information provided from the operation part 21 and the diagnostic results of the lesion diagnostic device 10. Upon receiving an input from the user made by selecting one of the buttons on the operation part 21, the controller 23 may generate a control signal for controlling the operation of the body part 22 to correspond to the selected button. For example, if the user selects the forward button for the body part 22, the controller 23 may generate an operation control signal to enable the body part 22 to move forward inside the patient's body at a constant speed. The body part 22 may move forward inside the patient's body based on a control signal from the controller 23.

Moreover, the controller 23 may generate a control signal for controlling the operation of the imaging part (not shown). The control signal for controlling the operation of the imaging part (not shown) may be a signal for allowing the imaging part (not shown) positioned in a lesion area to capture a gastric lesion image. In other words, if the user wants the imaging part (not shown) positioned in a specific lesion area to acquire an image based on an input from the operation part 21, they may click on a capture button. The controller 23 may generate a control signal to allow the imaging part (not shown) to acquire an image in the lesion area based on input information provided from the operation part 21. The controller 23 may generate a control signal for acquiring a specific lesion gastric image from the video the imaging part (not shown) is recording.

Additionally, the controller 23 may generate a control signal for controlling the operation of the biopsy unit for sampling a portion of tissue in the patient's body based on the diagnostic results of the lesion diagnostic device 10. If the diagnosis by the lesion diagnostic device 10 is classified as at least one of the following categories: advanced gastric cancer, early gastric cancer, high-grade dysplasia, and low-grade dysplasia, the controller 23 may generate a control signal for controlling the operation of the biopsy unit to perform an excision. The biopsy unit may include a variety of medical instruments, such as scalpels, needles, and so on, for sampling a portion of tissue from a living organism, and the scalpels and needles in the biopsy unit may be inserted into the body through a biopsy channel by the endoscopist to sample cells in the body. Also, the controller 23 may generate a control signal for controlling the operation of the biopsy unit based on a user input signal provided from the operation part 21. The user may perform the operation of sampling, excising, or removing cells inside the body by using the operation part 21.

According to an exemplary embodiment of the present disclosure, the lesion location acquisition part 24 may generate gastric lesion information by linking the gastric lesion images provided from the imaging part (not shown) with location information. The location information may be information on the current location of the body part 22 inside the body. In other words, if the body part 22 is positioned at a first point on the stomach of the patient's body and a gastric lesion image is acquired from the first point, the lesion location acquisition part 24 may generate gastric lesion information by linking this gastric lesion image with the location information.

The lesion location acquisition part 24 may provide the user (doctor) with the gastric lesion information generated by linking the acquired lesion gastric lesion images with the location information. By providing the user with the diagnostic results of the lesion diagnostic part 10 and the gastric lesion information of the gastric lesion location acquisition part 24 through the display 25, the risk of excision somewhere else other than the target lesion may be avoided when performing an excision treatment or surgery on the target lesion.

Moreover, if the biopsy unit is not positioned in the target lesion based on the location information provided from the lesion location acquisition part 24, the controller 23 may generate a control signal for controlling the position of the biopsy unit.

FIG. 3 is a schematic block diagram of a lesion diagnostic part of an endoscopic device according to an exemplary embodiment of the present disclosure.

Referring to FIG. 3, the lesion diagnostic part 10 may include an image acquisition part 11, a data generation part 12, a data preprocessing part 13, an artificial neural network building part 14, and a gastric lesion diagnostic part 15. However, the components of the lesion diagnostic part 10 are not limited to those disclosed above. For example, the lesion diagnostic part 10 may further include a database for storing information.

The image acquisition part 11 may acquire new gastric lesion images. The image acquisition part 11 may receive new gastric lesion images from an imaging part (not shown). The image acquisition part 11 may acquire new gastric lesion images acquired with an endoscopic video imaging device (digital camera) used for gastroendoscopy. The image acquisition part 11 may collect white-light gastroendoscopic images of a pathologically confirmed lesion. The new gastric lesion images may be gastric lesion images that are acquired in real time through an imaging part (not shown) in an endoscopic examination (treatment).

Moreover, the image acquisition part 11 may acquire images that are taken by varying either the angle, direction, or distance of a first area in the patient's stomach. The image acquisition part 11 may acquire new gastric lesion images in JPEG format. The new gastric lesion images may be styled with a 35-degree field of view at 1280×640 pixel resolution. Meanwhile, the image acquisition part 11 may acquire new gastric lesion images from which their individual identifier information has been removed. The image acquisition part 11 may acquire new gastric lesion images where the lesion is located at the center and where the black frame area has been removed.

On the contrary, if the image acquisition part 11 acquires images of low quality or low resolution, such as out-of-focus images, images including artifacts, and low-dynamic-range images, these images may be excluded. In other words, the image acquisition part 11 may exclude images if they are not applicable to a deep learning algorithm.

According to another exemplary embodiment of the present disclosure, the endoscopic device 1 may be a device that is made in capsule form. The capsule endoscopic device 1 may be inserted into the body of a patient and remotely operated. New gastric lesion images acquired from the capsule endoscopic device 1 may include all image data acquired by video recording, as well as images of a region the user wants to capture.

The data generation part 12 may generate a new dataset by linking new gastric lesion images with patient information. The patient information may include the patient's sex, age, height, weight, race, nationality, smoking status, alcohol intake, and family history. Furthermore, the patient information may include clinical information. The clinical information may refer to all data a doctor can use when making a specific diagnosis in a hospital. Particularly, the clinical information may include electronic medical records containing personal information like sex and age, specific medical treatments received, billing information, and orders and prescriptions, which are created throughout a medical procedure. Moreover, the clinical information may include biometric data such as genetic information. The biometric data may include personal health information containing numerical data like heart rate, electrocardiogram, exercise and movement levels, oxygen saturation, blood pressure, weight, and blood sugar levels.

The patient information is data that is fed into a fully-connected neural network, along with the output from the convolutional neural network architecture, from the artificial neural network building part 14 to be described later, and further improvements in accuracy can be expected by feeding other information other than gastric lesion images as input into an artificial neural network.

The preprocessing part 13 may preprocess a new dataset in a way that is applicable to a deep learning algorithm. The preprocessing part 13 may preprocess a new dataset in order to enhance the recognition performance of the deep learning algorithm and minimize similarities between different patients' images. The deep learning algorithm may be composed of two parts: a convolutional neural network architecture and a fully-connected neural network architecture.

According to an exemplary embodiment of the present disclosure, the preprocessing part 13 may perform a preprocessing process in five phases. First of all, the preprocessing part 13 may perform a cropping phase. In the cropping phase, an unnecessary portion (on a black background) on the edge around a lesion may be excluded from a new gastric lesion image acquired by the image acquisition part 11. For example, the preprocessing part 13 may cut the gastric lesion image to an arbitrarily specified pixel size (e.g., 299×299 pixels or 244×244 pixels). In other words, the preprocessing part 13 may cut the new gastric lesion image to a size applicable for the deep learning algorithm.

Next, the preprocessing part 13 may perform a parallel shifting phase. The preprocessing part 13 may shift the new gastric lesion image in parallel upward, downward, to the left, or to the right. Also, the preprocessing part 13 may perform a flipping phase. For example, the preprocessing part 13 may flip the gastric lesion image vertically. Also, the preprocessing part 13 may flip the gastric lesion image upward or downward and then flip it to the left or right.

Moreover, the preprocessing part 13 may perform a color adjustment phase. For example, in the color adjustment phase, the preprocessing part 13 may perform color adjustment on an image based on colors extracted by computing the mean RGB values across the entire dataset and subtracting them from the image. Also, the preprocessing part 13 may randomly adjust colors in the new gastric lesion image.

The preprocessing part 13 may generate a dataset of new gastric lesion images applicable to the deep learning algorithm by performing the five phases of the preprocessing process. Also, the preprocessing part 13 may generate a dataset of new gastric lesion images applicable to the deep learning algorithm by performing at least one of the five phases of the preprocessing process.

Furthermore, the preprocessing part 13 may perform a resizing phase. The resizing phase may be a phase in which a gastric lesion image is enlarged or reduced to a preset size.

The preprocessing part 13 may include an augmentation part (not shown) for augmenting image data to increase the amount of new gastric lesion image data.

According to an exemplary embodiment of the present disclosure, in the case of a deep learning algorithm including a convolutional neural network, the greater the amount of data, the better the performance. However, the amount of new gastroendoscopic images from endoscopic examinations is much less than the amounts of images from other types of examinations, and therefore the volume of new gastric lesion image data collected and acquired by the image acquisition part 11 may be very insufficient for use on a convolutional neural network. The augmentation part (not shown) may perform a data augmentation process by applying at least one of the following: rotating, flipping, cropping, and adding noise into new gastric lesion images.

The preprocessing part 13 may perform a preprocessing process in a way that corresponds to a preset reference value. The preset reference value may be arbitrarily specified by the user. Also, the preset reference value may be determined by an average value for acquired new gastric lesion images. A new dataset may be provided to the artificial neural network building part 14 once it has undergone the preprocessing part 13.

Hereinafter, an example of building an artificial neural network system by the artificial neural network building part 14 will be described.

According to an exemplary embodiment of the present disclosure, the artificial neural network system 14 may build an artificial neural network system based on a dataset that is generated by acquiring a plurality of gastric lesion images by the image acquisition part 11 and linking data of the gastric lesion images with patient information by the data generation part 12.

The artificial neural network building part 14 may build an artificial neural network system by using a plurality of gastric lesion images the image acquisition part 11 has received from a plurality of hospitals' image storage devices and database systems. The plurality of hospitals' image storage devices may be devices that store gastric lesion images acquired during gastroendoscopy in multiple hospitals.

Moreover, the artificial neural network building part 14 may undergo a process of preprocessing the dataset in a way that is applicable to a deep learning algorithm. The preprocessing process may be performed by the above-described data preprocessing part 13. For example, the artificial neural network building part 14 may preprocess the dataset such that it becomes applicable to a deep learning algorithm by passing the gastric lesion images in the dataset through the five phases of the preprocessing process performed by the above-described preprocessing part 13.

For example, the data generation part 12 may generate a training dataset and a validation dataset, for use on a deep learning algorithm. A dataset, when generated, may be classified as a training dataset required for training the artificial neural network or a validation dataset for validating information on the progress of the training of the artificial neural network.

In addition, the data generation part 12 may classify a plurality of gastric lesion images acquired by the image acquisition part 11 into images to be randomly used for a training dataset and images used for a validation dataset. Also, the data generation part 12 may use all other images, except for those used for the validation dataset, as the training dataset. The validation dataset may be randomly selected. The percentage of the validation dataset and the percentage of the training dataset may take on preset reference values. The preset reference values may be 10% for the validation dataset and 90% for the training dataset, respectively, but not limited thereto.

The data generation part 12 may generate the training dataset and the validation dataset separately in order to avoid overfitting. For example, neural network architectures may be overfitted to the training dataset due to their learning characteristics. Thus, the data generation part 12 may use the validation dataset to avoid overfitting of the artificial neural network.

The validation dataset may be a dataset that is not redundant with the training dataset. Since validation data is not used for building an artificial neural network, the validation data is the first data that the artificial neural network will encounter during validation. Accordingly, the validation dataset may be suitable for evaluating the performance of the artificial neural network when new images (not used for training) are fed as input.

The artificial neural network building part 14 may build an artificial neural network by training the artificial neural network by using a preprocessed dataset as input and gastric lesion classification results as output.

According to an exemplary embodiment of the present disclosure, the artificial neural network building part 14 may provide gastric lesion classification results as output by applying a deep learning algorithm consisting of two parts: a convolutional neural network architecture and a fully-connected neural network architecture. The fully-connected neural network is a neural network in which nodes are two-dimensionally interconnected horizontally and longitudinally and there are interconnections between nodes on adjacent layers but not between nodes within the same layer.

The artificial neural network building part 14 may build a training model in which a convolutional neural network is trained by taking a preprocessed training dataset as input and a fully-connected neural network is trained by taking the output of the convolutional neural network as input.

According to an exemplary embodiment of the present disclosure, the convolutional neural network may extract a plurality of specific feature patterns by analyzing gastric lesion images. The extracted specific feature patterns may be used for final classification in the fully-connected neural network.

Convolutional neural networks are a type of neural network mainly used for speech recognition or image recognition. Since the convolutional neural network is constructed to process multidimensional array data, it is specialized for processing a multidimensional array such as a color image array. Accordingly, most techniques using deep learning in image recognition are based on convolutional neural networks.

The convolutional neural network CNN processes an image by partitioning it into multiple segments, rather than using the whole image as a single piece of data. This can extract local features of the image even if the image is distorted, thereby allowing the convolutional neural network CNN to deliver proper performance.

The convolutional neural network may consist of a plurality of layers. The elements of each layer may include a convolutional layer, an activation function, a max pooling layer, an activation function, and a dropout layer. The convolutional layer serves as a filter called a kernel to locally process the entire image (or a newly generated feature pattern) and extract a new feature pattern of the same size as the image. For a feature pattern, the convolutional layer may correct the values of the feature pattern through the activation function to make it easier to process them. The max pooling layer may take a sample from a gastric lesion image and reduce the size of the image by size adjustment. Although feature patterns are reduced in size as they pass through the convolutional layer and the max pooling layer, the convolutional neural network may extract a plurality of feature patterns by using a plurality of kernels. The dropout layer may involve a method in which, when training the weights of the convolutional neural network, some of the weights are not used deliberately for efficient training. Meanwhile, the dropout layer may not be applied when actual testing is performed through a training model.

A plurality of feature patterns extracted from the convolutional neural network may be delivered to the following phase, i.e., the fully-convolutional neural network, and used for classification. The convolutional neural network may adjust the number of layers. By adjusting the number of layers in the convolutional neural network to fit the amount of training data required for model training, the model can be built with higher stability.

Moreover, the artificial neural network building part 14 may build a diagnostic (training) model in which a convolutional neural network is trained by taking a preprocessed training dataset as input and a fully-connected neural network is trained by taking the output of the convolutional neural network and the patient information as input. In other words, the artificial neural network building part 14 may allow preprocessed image data to preferentially enter the convolutional neural network and allow the output of the convolutional neural network to enter the fully-connected neural network. Also, the artificial neural network building part 14 may allow randomly extracted features to directly enter the fully-connected neural network without passing through the convolutional neural network.

In this case, the patient information may include various information such as the patient's sex, age, height, weight, race, nationality, smoking status, alcohol intake, and family history. Furthermore, the patient information may include clinical information. The clinical information may refer to all data a doctor can use when making a specific diagnosis in a hospital. Particularly, the clinical information may include electronic medical records containing personal information like sex and age, specific medical treatments received, billing information, and orders and prescriptions, which are created throughout a medical procedure. Moreover, the clinical information may include biometric data such as genetic information. The biometric data may include personal health information containing numerical data like heart rate, electrocardiogram, exercise and movement levels, oxygen saturation, blood pressure, weight, and blood sugar levels.

The patient information is data that is fed into a fully-connected neural network, along with the output of the convolutional neural network architecture, from the artificial neural network building part 14, and further improvements in accuracy can be expected by feeding the patient information as input into an artificial neural network, rather than deriving the output by using gastric lesion images alone.

For example, once training is done on clinical information in a training dataset, indicating that the incidence of cancer increases with age, the input of an age 42 or 79, along with image features, may derive gastric lesion classification results showing that older patients with an uncertain lesion that is hard to classify as benign or malignant have a higher probability of cancer.

The artificial neural network building part 14 may perform training by applying training data to a deep learning algorithm architecture (an architecture in which the training data is fed into the fully-connected neural network through the convolutional neural network), calculating the error between the output derived from the training data and the actual output, and giving feedback on the outputs through a backpropagation algorithm to gradually change the weights of the neural network architecture by an amount corresponding to the error. The backpropagation algorithm may adjust the weight between each node and its next node in order to reduce the output error (difference between the actual output and the derived output). The artificial neural network building part 14 may derive a final diagnostic model by training the neural networks on a training dataset and a validation dataset and calculating weight parameters.

The gastric lesion diagnostic part 15 may perform a gastric lesion diagnosis through an artificial neural network after passing a new dataset through a preprocessing process. In other words, the gastric lesion diagnostic part 15 may derive a diagnosis on new gastroendoscopic images by using the final diagnostic model derived by the artificial neural network building part 14.

The new gastroendoscopic images may be real-time gastroendoscopic images acquired by the imaging part of the endoscopic device 1. The new gastroendoscopic images may include gastric lesion images based on which the user wants to make a diagnosis. The new dataset may be a dataset that is generated by linking gastric lesion images with patient information. The new dataset may be preprocessed such that it becomes applicable to a deep learning algorithm after passing through the preprocessing process of the preprocessing part 13. Afterwards, the preprocessed new dataset may be fed into the artificial neural network building part 14 to make a diagnosis with respect to the gastric lesion images based on training parameters.

According to an exemplary embodiment of the present disclosure, the gastric lesion diagnostic part 15 may classify a gastric lesion diagnosis as at least one of the following categories: advanced gastric cancer, early gastric cancer, high-grade dysplasia, and low-grade dysplasia. Moreover, the gastric lesion diagnostic part 15 may diagnose and classify gastric lesions as cancerous or non-cancerous. Also, the gastric lesion diagnostic part 15 may diagnose and classify gastric lesions into two categories: neoplasm and non-neoplasm. The neoplasm category may include AGC, EGC, HGD, and LGD. The non-neoplasm category may include lesions such as gastritis, benign ulcers, erosions, polyps, or intestinal metaplasia, and epithelial tumor.

The lesion diagnostic part 10 may analyze images acquired by the imaging part (not shown) and automatically classify and diagnose uncertain lesions, in order to reduce side effects of an unnecessary biopsy or endoscopic excision performed to classify and diagnose uncertain lesions, and may generate information to allow the doctor to proceed with an endoscopic excision treatment by using a plurality of units included in the body part 22 in the case of a neoplasm (dangerous tumor).

Hereinafter, the operation flow of the present disclosure will be discussed briefly based on what has been described in detail above.

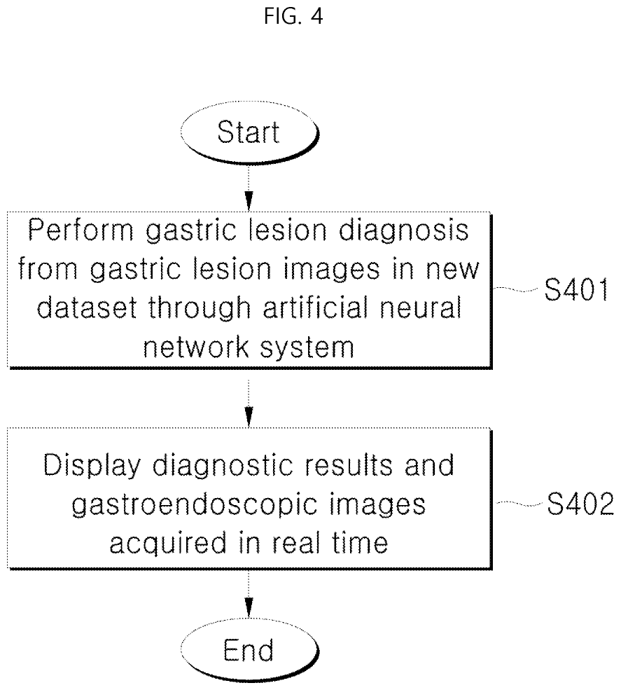

FIG. 4 is an operation flow chart of a method for diagnosing a lesion from gastroendoscopic images acquired in real time by an endoscopic device according to an exemplary embodiment of the present disclosure.

The method for diagnosing a lesion from gastroendoscopic images acquired in real time by an endoscopic device, shown in FIG. 4, may be performed by the above-described endoscopic device 1. Thus, the description of the endoscopic device 1 may be omitted since it may apply equally to the method for diagnosing a lesion from gastroendoscopic images acquired in real time by an endoscopic device.

In the step S401, the endoscopic device 1 may perform a gastric lesion diagnosis from gastric lesion images in a new dataset through an artificial neural network. The endoscopic device 1 may acquire a plurality of gastric lesion images prior to the step S401. The gastric lesion images may be white-light images. Also, the endoscopic device 1 may generate a dataset by linking a plurality of gastric lesion images with patient information. When generating a dataset, the endoscopic device 1 may classify the dataset as a training dataset required for training the artificial neural network or a validation dataset for validating information on the progress of the training of the artificial neural network. In this case, the validation dataset may be a dataset that is not redundant with the training dataset. The validation dataset may be used for evaluating the performance of the artificial neural network when a new dataset is fed as input into the artificial neural network after passing through the preprocessing process.

Moreover, the endoscopic device 1 may preprocess a new dataset in a way that is applicable to a deep learning algorithm. The endoscopic device 1 may perform a cropping process in which a peripheral area of a new gastric lesion image included in the new dataset is cropped around the gastric lesion to a size applicable for the deep learning algorithm in such a way that the gastric lesion is not included in the image. Also, the endoscopic device 1 may shift the new gastric lesion image in parallel upward, downward, to the left, or to the right. Also, the endoscopic device 1 may flip the new gastric lesion image. Also, the endoscopic device 1 may adjust colors in the new gastric lesion image. The endoscopic device 1 may preprocess the new gastric lesion image in a way that is applicable to the deep learning algorithm.

Moreover, the endoscopic device 1 may augment image data to increase the amount of new gastric lesion image data. The endoscopic device 1 may augment new gastric lesion image data by applying at least one of the following: rotating, flipping, cropping, and adding noise into the gastric lesion image data.

The endoscopic device 1 may build an artificial neural network by training the artificial neural network by using a preprocessed dataset as input and gastric lesion classification results as output. The endoscopic device 1 may build a training model in which a convolutional neural network and a fully-connected neural network are trained by using the preprocessed dataset as input and the gastric lesion classification results of the convolutional neural network as output.

In addition, the endoscopic device 1 may build a training model in which a convolutional neural network is trained by taking a preprocessed dataset as input and a fully-connected neural network is trained by taking the output of the convolutional neural network and the patient information as input. The convolutional neural network may output a plurality of feature patterns from a plurality of gastric lesion images, and the plurality of feature patterns may be finally classified by the fully-connected neural network.

The endoscopic device 1 may perform a gastric lesion diagnosis through the artificial neural network after passing a new dataset through the preprocessing process. The endoscopic device 1 may classify a gastric lesion diagnosis based on new gastroendoscopic images as at least one of the following categories: advanced gastric cancer, early gastric cancer, high-grade dysplasia, and low-grade dysplasia.

In the step S402, the endoscopic device 1 may output new gastroendoscopic images acquired in real time and gastric lesion diagnostic results produced through the artificial neural network.

In the above description, the steps S401 and S402 may be further subdivided into a greater number of steps or combined into a smaller number of steps in some examples of implementation of the present disclosure. Moreover, some of the steps may be omitted if necessary, or the sequence of the steps may be changed.

A method for diagnosing a gastric lesion from gastroendoscopic images acquired in real time by an endoscopic device according to an exemplary embodiment of the present disclosure may be realized in the form of program instructions which can be implemented through various computer components, and may be recorded in a computer-readable storage medium. The computer-readable storage medium may include program instructions, a data file, a data structure, and the like either alone or in combination thereof. The program instructions recorded in the computer-readable storage medium may be any program instructions particularly designed and structured for the present disclosure or known to those skilled in the field of computer software. Examples of the computer-readable storage medium include magnetic recording media, such as hard disks, floppy disks and magnetic tapes, optical data storage media, such as CD-ROMs and DVD-ROMs, magneto-optical media such as floptical disks, and hardware devices, such as read-only memories (ROMs), random-access memories (RAMs), and flash memories, which are particularly structured to store and implement the program instructions. Examples of the program instructions include not only assembly language code formatted by a compiler but also high-level language code which can be implemented by a computer using an interpreter. The hardware device described above may be configured to operate as one or more software modules to perform operations of the present disclosure, and vice versa.

In addition, the above-described method for diagnosing a gastric lesion from gastroendoscopic images acquired in real time by an endoscopic device also may be implemented in the form of a computer-executable computer program or application stored in a recording medium.

Although some embodiments have been described herein, it should be understood that these embodiments are provided for illustration and that various modifications, changes, alterations, and equivalent embodiments can be made by those skilled in the art without departing from the spirit and scope of the present disclosure. Therefore, the embodiments are not to be construed in any way as limiting the present disclosure. For example, each component described as a single type may be implemented in a distributed manner, and, similarly, components described as distributed may be implemented in a combined form.

The scope of the present application should be defined by the appended claims and equivalents thereof rather than by the detailed description, and all changes or modifications derived from the spirit and scope of the claims and equivalents thereof should be construed as within the scope of the present disclosure.

1. An endoscopic device for diagnosing a gastric lesion from gastroendoscopic images acquired in real time, the endoscopic device comprising:
a body part containing a plurality of units, that is inserted into a patient's body;
an operation part provided on the rear end of the body part, for manipulating the body part based on user input information;
a lesion diagnostic part that builds an artificial neural network system by training the artificial neural network system by using a plurality of gastric lesion images as input and gastric lesion diagnostic results as output, generates a new dataset by linking new gastroendoscopic images acquired in real time with patient information, and performs a gastric lesion diagnosis through the built artificial neural network system; and
a display that shows the diagnostic results of the lesion diagnostic part and the new gastroendoscopic images acquired in real time.

2. The endoscopic device of claim 1, further comprising a controller that generates a control signal for controlling the operation of the body part based on the user input information provided from the operation part and the diagnostic results of the lesion diagnostic device.

3. The endoscopic device of claim 2, wherein the body part comprises an imaging part provided on the front end of the body part, that takes new gastric lesion images and provides the taken, new gastroendoscopic images to the lesion diagnostic part,
wherein the controller receives the user's input for controlling the operation of the imaging part from the operation part and generates a control signal for controlling the imaging part.

4. The endoscopic device of claim 3, further comprising a lesion location acquisition part that generates gastric lesion information by linking the new gastroendoscopic images provided from the imaging part with location information,
wherein the controller generates a control signal for controlling the operation of a biopsy unit for sampling a portion of tissue in the patient's body based on the diagnostic results of the lesion diagnostic part and the gastric lesion information.

5. The endoscopic device of claim 3, wherein the lesion diagnostic part comprises:
an image acquisition part for acquiring the new gastric lesion images;
a data generation part for generating a new dataset by linking the new gastric lesion images with the patient information;
a data preprocessing part for preprocessing the new dataset in a way that is applicable to a deep learning algorithm;
an artificial neural network building part for building an artificial neural network system by training the artificial neural network system by using a plurality of gastric lesion images as input and gastric lesion diagnostic results as output; and
a gastric lesion diagnostic part that performs a gastric lesion diagnosis through the artificial neural network system after passing the new dataset through a preprocessing process.

6. The endoscopic device of claim 5, wherein the data generation part generates a dataset by linking each of the plurality of gastric lesion images with the patient information, and the dataset, when generated, is classified as a training dataset required for training the artificial neural network system or a validation dataset for validating information on the progress of the training of the artificial neural network system.

7. The endoscopic device of claim 6, wherein the validation dataset is a dataset that is not redundant with the training dataset.

8. The endoscopic device of claim 5, wherein the preprocessing part preprocesses a gastric lesion image in the new dataset in a way that is applicable to the deep learning algorithm by performing at least one of the following phases of the preprocessing process: cropping a peripheral area of the gastric lesion image around the gastric lesion in such a way that the gastric lesion is not included in the image; shifting the gastric lesion image in parallel upward, downward, to the left, or to the right; rotating the gastric lesion image; flipping the gastric lesion image; and adjusting colors in the gastric lesion image.

9. The endoscopic device of claim 8, wherein the preprocessing part comprises an augmentation part for increasing the amount of new gastric lesion image data,
wherein the augmentation part augments the new gastric lesion image data by applying at least one of the following: rotating, flipping, cropping, and adding noise into the gastric lesion image data.

10. The endoscopic device of claim 6, wherein the artificial neural network building part builds a training model in which a convolutional neural network and a fully-connected neural network are trained by using the preprocessed dataset as input and the gastric lesion diagnostic results as output.

11. The endoscopic device of claim 10, wherein the preprocessed dataset is fed as input into the convolutional neural network, and the output of the convolutional neural network and the patient information are fed as input into the fully-connected neural network.

12. The endoscopic device of claim 11, wherein the convolutional neural network produces a plurality of feature patterns from the plurality of gastric lesion images,
wherein the plurality of feature patterns are finally classified by the fully-connected neural network.

13. The endoscopic device of claim 5, wherein the gastric lesion diagnostic part classifies the gastric lesion diagnosis as at least one of the following categories: advanced gastric cancer, early gastric cancer, high-grade dysplasia, and low-grade dysplasia.

14. A method for diagnosing a gastric lesion from gastroendoscopic images acquired in real time by an endoscopic device,
the endoscopic device comprising: a body part to be inserted into a patient's body; and an operation part provided on the rear end of the body part, for manipulating the body part based on user input information,
the method comprising:
building an artificial neural network system by training the artificial neural network system by using a plurality of gastric lesion images as input and gastric lesion diagnostic results as output, generating a new dataset by linking new gastroendoscopic images with patient information, and performing a gastric lesion diagnosis through the built artificial neural network system; and
showing the diagnostic results and the new gastroendoscopic images acquired in real time.

15. A computer-readable recording medium storing a program for executing the method of claim 14.
